(19) United States
(12) Patent Application Publication
(10) Pub. No.: US 2019/0343593 A1
(43) Pub. Date: Nov. 14, 2019

(54) Intervention Guidance Device

(71) Applicant: Norwegian University of Science and Technology (NTNU), Trondheim (NO)

(72) Inventor: Daniel Fossum Bratbak, Trondheim (NO)

(21) Appl. No.: 16/522,935

(22) Filed: Jul. 26, 2019

Related U.S. Application Data
(63) Continuation of application No. 15/537,313, filed on Jun. 16, 2017.

(30) Foreign Application Priority Data
Dec. 18, 2014 (GB) 1422551.0

(51) Int. Cl. A61B 90/11; A61B 17/34; A61B 34/20; A61B 90/16

(52) U.S. Cl. CPC A61B 90/11; A61B 17/3403; A61B 17/3472; A61B 2090/363; A61B 90/16; A61B 2034/2057; A61B 2017/3407; A61B 34/20

(57) ABSTRACT

A guidance device for guidance of surgical interventions on a patient, the surgical interventions requiring an intervention device to surgically enter the body and be directed through body tissues to a target site within the patient's head, the guidance device comprising: a guide piece 4 for guiding the intervention device and directing it to the target site within the patient's head; a mouthpiece 2 arranged to anchor the device in a fixed orientation relative to the patient's upper jaw or lower jaw; and a targeted or targetable mounting 6 supporting the guide piece on the mouthpiece, the mounting 6 being for directing the guide piece 4 in a desired orientation relative to the mouthpiece 2 to thereby direct the intervention device through body tissues to the target site in the patient's head.

### CROSS-REFERENCE TO RELATED APPLICATIONS

This application is a continuation of U.S. application Ser. No. 15/537,313, filed Jun. 16, 2017, which is a U.S. National Stage application under 35 U.S.C. § 371 of International Application PCT/EP2015/079989 (published as WO/2016/096984 A1), filed Dec. 16, 2015, which claims priority to GB Application No. 1422551.0 filed Dec. 18, 2014. Benefit of the filing date of this prior application is hereby claimed. This prior application is hereby incorporated by reference in its entirety.

The invention relates to a guidance device for guiding surgical interventions within the head, for example for guidance of an injection of a substance into a target site in the head. In one example the device is used for guidance of injections towards cranial parasympathetic ganglia. The invention also relates to the use of such a device in the treatment of medical conditions, for example in the treatment of primary headaches.

Migraine is a primary headache that may be characterized as a unilateral headache associated with symptoms like nausea, photophobia and phonophobia. More than 50% have as well cranial autonomic symptoms such as lacrimation, conjunctival injection, nasal congestion and rhinorrhoea.

A possible mechanism for a migraine attack is parasympathetic activation with nitrogen oxide (NO) as transmitter inducing dilatation of cranial blood vessels, plasma protein extravasation and release of inflammatory substances. The catalysing enzyme for NO, NOS (NO synthases), has been located in perivascular nerve fibres on cerebral arteries and traced back to the sphenopalatine ganglion (SPG) and otic ganglion (OG), as described by Olesen J. in “The role of nitric oxide (NO) in migraine, tension-type headache and cluster headache”, Pharmacology and Therapeutics, 2008; 120; 157-171.

Blocking of the SPG by application of lidocaine has shown to be effective in randomised, controlled studies of acute treatment of migraine (see Maizels M, Scott B, Cohen W and Chen W, “Intranasal lidocaine for treatment of migraine: a randomized, double-blind, controlled trial” JAMA, 1996; 276(4):319-21 and Maizels M and Geiger A M, “Intranasal lidocaine for migraine: a randomized trial and open-label follow-up”, Headache, 1999; 39(8):543-51). Blocking via botulinum toxin is also described in the prior art, for example in U.S. Pat. No. 7,981,433.

The trigeminal autonomic cephalalgias (TACs) are a group of primary headache disorders characterized by unilateral head pain that occurs in association with ipsilateral cranial autonomic features such as lacrimation, conjuctival injection and nasal symptoms. The TACs include hemicrania continua, paroxysmal hemicrania, short lasting unilateral neuralgiform headache with conjunctival injection and tearing/cranial autonomic features (SUNCT/SUNA) and cluster headache.

Cluster headache is a severe unilateral headache associated with ipsilateral autonomic symptoms and characterised by a circannual and circadian periodicity (see Goadsby P J, Cittadini E, Burns B and Cohen A, “Trigeminal autonomic cephalalgias: diagnostic and therapeutic developments” Curr Opin Neurol, 2008; 21:323-330). Approximately 90% suffer from the episodic form and 10% from the chronic form. Based on functional neuroimaging central to the pathophysiology of the disease may be an abnormality in hypothalamic function that facilitate a cascade of metabolic and other biochemical events triggering an attack (see Cohen A S and Goadsby P J, “Functional neuroimaging of primary headache disorders” Expert Rev Neurother, 2006; 6(8):1159-1171). This sets off a positive feedback system involving the trigeminovascular system as the afferent limb and the parasympathetic outflow from the superior salivatory nucleus via the facial nerve through the SPG and OG as the efferent limb (see Goadsby P J, “Pathophysiology of cluster headache: a trigeminal autonomic cephalgia” Lancet Neurol. 2002; 1:251-57). Thus, vasodilatation of the pain-producing large cranial vessels and dura mater starts a reflex activation of parasympathetic vasodilator efferents which activate the trigeminal endings further to produce the excruciating pain and the parasympathetic symptoms (lacrimation and nasal congestion/secretion) seen in cluster headaches. In addition, the carotid swelling leads to a neuropraxic lesion of the sympathetic plexus surrounding the artery, resulting in a partial ipsilateral Homer's syndrome (ptose, miosis and conjunctival injection).

Current strategies for surgical treatment of these headaches include neurodestructive procedures targeting the trigeminal system (afferent limb) and the SPG (efferent limb), and neurostimulating procedures targeting the great occipital nerve and grey matter of hypothalamus (deep brain stimulation, DBS). Thus, cranial autonomic ganglia, and especially SPG and OG, are thought to have a role in the development of primary headaches and treatments have been established targeting the SPG.

Primary headaches may be hard to treat and the need for preventive treatments is enormous. Apart from CGRP antagonism, inhibition of the NO pathway may be considered the best documented and most promising target for treatment of primary headache (as described by Olesen J. in the reference above).

The trigeminal nerve is involved in all types of headache, including secondary headaches, i.e. headaches caused by other pathologies.

Sinonasal polyposis is a chronic hyperplastic disease of the nasal mucosa and the paranasal sinuses. There is a well established association between polyposis and rhinitis. The causes underlying the association could be due to chronic inflammation most likely induced by unstable autonomous nerve control of nasal vasomotor activity. This may precede the occurrence of nasal polyps. Vasomotor rhinitis seems to be related to an imbalance in the cranial autonomic system between parasympathetic and sympathetic activity. Therapies include vidianectomi and other forms of autonomic denervation which blocks parasympathetic activity through the SPG. Vidianectomi and other forms of autonomic denervation have also been an option for treating allergic rhinitis and new modified surgical techniques yield optimistic results.

Blocking the parasympathetic activity passing through the SPG by vidian neurectomy has shown to be effective in allergic rhinitis (see Wan-Fu SU, Shao-Cheng Liu, Feng-Shiang Chiu and Chia-Hsuan Lee. Antegrade transsphenoidal vidian neurectomy: Short-term surgical outcome analysis. Am J Rhinol Allergy 2011; 25:e217-e220), vasomotor rhinitis and rhinosinusitis with polyposis (see Cassano M, Mariano G, Russo L, Cassano P. Sphenopalatine artery ligation with nerve resection in patients with vasomotor rhinitis and polyposis: a prospective, randomized, double-blind investigation. Acta Oto-Laryngologica 2012; 132(5):525-32).

Almost all patients who undergo parotidectomy will to some extent develop Frey syndrome (auriculotemporal syndrome or gustatory sweating) after surgery, because of aberrant regeneration of cut parasympathetic fibres between otic ganglion and subcutaneous vessels. Frey syndrome may also occur after extirpation of the submandibular gland, mandibular condylar fracture, and obstetric trauma caused by forceps. Nontraumatic causes are sympathectomy, autonomic neuropathy in diabetes mellitus, herpes zoster infection, and metabolic diseases. Frey syndrome may cause considerable social embarrassment and social incapacity due to profuse flushing and sweating when eating. Blocking the parasympathetic activity through the OG may constitute an effective treatment for these patients.

The cranial autonomic ganglia, and especially the SPG and the OG, are hence interesting targets for treating such entities, but they are not easily reached for interventions such as infiltration with pharmacological substances, destructive procedures or neuromodulation.

There are four paired cranial parasympathetic ganglia: sphenopalatine (pterygopalatine) ganglion (SPG), otic ganglion (OG), ciliary ganglion, and submandibular ganglion.

The SPG is pyramid shaped with a mean diameter of 3.5 mm. It is suspended from the maxillary nerve by the sphenopalatine nerves. Preganglionic parasympathetic fibres form the nervus intermedius of the facial nerve synapse with postganglionic fibres innervating the lacrimal gland, mucosa of the sinonasal cavity and cerebral blood vessels. Postganglionic sympathetic fibres from the superior cervical ganglion pass through the ganglion as well as sensory nerves from the maxillary nerve that innervates the palate and the epipharynx. The SPG can be identified using MRI.

The SPG is situated in the sphenopalatine (pterygopalatine) fossa (SF) and has the shape of a funnel flattened in the coronal plane. It is wider superiorly and then narrows down inferiorly with the apex pointing downwards into the greater palatine canal. SF has the following boundaries; superiorly with the infraorbital fissure, laterally with the pterygomaxillary fissure, medially with the palatine bone, posteriorly with the pterygoid plates, anteriorly with the posterior wall of the maxillary sinus and inferiorly with the palatine canal. Additionally, it communicates with the nasal cavity through the sphenopalatine foramen and the middle cranial fossa through the vidian canal and foramen rotundum. It can be divided in three compartments, an anterior compartment containing mainly blood vessels, a middle compartment containing mainly adipose tissue, and a posterior compartment containing mainly neural structures.

The maxillary artery enters the SF through the pterygomaxillary fissure and branches into the sphenopalatine artery, descending palatine artery, infraorbital artery, alveolar arteries and the artery of the pterygoid canal. The SF is often devoid of endoscopic identifiable veins. Blood vessels of the SF are tightly packed as they loop the anterior compartment and therefore a lateromedial intervention is more likely to cause a bleeding than an anteroposterior approach.

The average distance from the SPG to the vidian canal is 2 7 mm, to the infraorbital fissure 20.3 mm and to foramen rotundum 4.7 mm It is normally located in the same vertical and horizontal plane as the vidian canal and posteriorly for the sphenopalatine foramen. The sphenopalatine foramen is vertically orientated located in the superomedial corner of SF with a diameter of 5-6 mm and typically located below the posterior end of the line of attachment of the middle turbinate and crista ethmoidalis, but this may vary. The average distance from the piriform aperture is 48 mm with an angle of elevation from the nasal floor is 22 degrees.

Such information of the distances from SPG to landmark identifiable on CT may be used to mark the SPG for image-guided interventions when MRI is contraindicated or not available.

The oral cavity communicates with the sphenopalatine fossa through the greater traspalatinal canal. The inferior opening is situated on the medial side of the second molar and the length of the canal is on average 25 mm. The canal transmits the descending palatine artery and vein, and the greater and lesser palatine nerves.

The OG is an oval structure measuring approximately 4 mm×3 mm×1.5 mm It is composed of parasympathetic fibres arising in the inferior salivatory nucleus in the medulla, sympathetic fibres form the superior cervical sympathetic ganglion, and motor fibres from the mandibular branch of the trigeminal nerve. The OG supplies secretory fibres to the parotid gland and parasympathetic fibre to cerebral blood vessels. It is situated just posterior of the lateral pterygoid plate below the foramen ovale in the infratemporal fossa and adjacent to the middle meningeal artery, mandibular nerve and buccal nerve.

For minimally invasive interventions in the SF there are three surgical approaches, each with its advantages and disadvantages; a lateral approach through the pterygomaxillary fissure, a medial transnasal approach through the sphenopalatine foramen and a transoral approach through the greater palatine canal. All approaches give a relatively easy access to SF for someone skilled to the art, but there are pivotal differences if a high-precision intervention in the closest proximity of the SPG is needed.

Image guided surgery (IGS) was developed to improve accuracy and precision. Such technology is used to assist in orientation by displaying the position of a pointer or surgical instrument on a medical image. Armless systems may be based on light, sound waves or magnetic fields. With the use of a computer platform, a tracking system and a body marker, a pointer or other instrument can be calibrated so that the navigation system will display the tip of the instrument correctly. The instruments are calibrated in advance by the manufacturer or the surgeon may use a universal instrument integration system to calibrate basically any instrument. This system is based on a set of universal clamps attached to the instrument. There are several limitations to this solution. Firstly, attaching the clamps can be challenging and they can easily move, hence giving a wrong impression of the actual localization of the instrument on the medical image. Secondly, semi-rigid instruments are not suitable for calibration because they can bend after calibration, such as e.g. a thin needle or a long forceps.

The lateral approach is typically carried out under local anaesthesia. Typically a high-precision intervention would be an lateral approach. Using the lateral approach there is a straight line through soft tissue from the skin to the SF, SPG, orbita and the sphenopalatine foramen. The distance from the skin to the SF or the SPG is approximately 6 cm making it next to impossible to achieve a high precision infiltration without the use of IGS. Violating the sphenopalatine foramen could result in a complicated posterior epistaxis, violating the infraorbital fissure could damage intraorbital tissue. Using the suprazygomatic approach, which is described in U.S. Pat. No. 7,981,433, for example, the sphenoid bone will normally obstruct access to the SF and in particular the middle and the posterior compartment and almost always obstruct access to the SPG, making it quite safe, but not applicable for high-precision interventions. If anatomical variations enable advancing a needle to the close proximity of the SPG by a suprazygomatic approach, it would be next to impossible to successfully target such a small structure with a conventional injection technique as described in U.S. Pat. No. 7,981,433. Due to the low diffusion rate of botulinum toxin and the fact that the SF mainly contains adipose tissue, a hydrophilic substance injected using these techniques will rarely reach its target.

The medial transnasal approach is difficult to perform under local anaesthesia due to the sensible posterior region of the nasal cavity, and the use of general anaesthesia makes it much less accessible. Due to the complex sinonasal anatomy it is normally performed by a rhinologist. For someone skilled in the art this approach may be the most accurate, mainly due to the low distance between the puncture site and the SPG. Normally such an approach is done by advancing the needle through the sphenopalatine foramen, risking damage to the sphenopalatine artery/arteries. The palatine bone, which constitutes the anterior border of the sphenopalatine foramen, is quite thin, and a suitable needle can quite easily be advanced through the bone, avoiding possible damage to the sphenopalatine artery.

However, such a procedure can easily bend the needle used, which will generally be an 18 G needle or thinner After it has been advanced through the bone the end of the needle is in the soft tissue and there is no way to know if deformation has occurred or to what extent, making the intervention unsafe and imprecise, with the use of IGS or not. For injections in deep tissue a 25 G needle or thinner is recommended to avoid unnecessary tissue damage, including bleedings and nerve damage. Furthermore, the thicker the needle the bigger the dead space, which hinders use of small injection volumes. As a consequence of these issues, needles suitable for SPG injection using the medial approach and also other approaches are not suitable for high-precision injections.

The transoral approach can be done with local anaesthesia. However, due to the direction of the palatine canal towards the very anterior part of the SF, high-precision interventions targeting the SPG are currently not feasible with this approach.

Intervention targeting the OG can be done via a lateral approach as described in interventions targeting the trigeminal ganglion through the oval foramen, or lateral approaches with the same injection sites as described above, i.e. lateral or suprazygomatic. It is also possible to apply a transnasal medial approach through the maxillary ostium and the posterior wall of the maxillary sinus and advancing adjacent to the lateral pterygoid plate. With this transnasal medial approach one can avoid important nerves and blood vessels in the infratemporal fossa and was performed without complications or side effects. This medial approach seems as well appropriate for neurostimulators as it can be situated and anchored to the pterygoid plate.

The cranial parasympathetic ganglia including the SPG and OG are surrounded by critical neural structures and organs like e.g. brain and eyes. Drug impact of these structures can cause serious complications and should be avoided. In addition, some medications diffuse slowly and they must be injected with millimetre accuracy to reach their target. As a result, accuracy is important in various situations:

1) When using a drug or implant that only works exactly where it is injected/situated.

2) Use of a diffusible drug that must be injected at a safe distance from sensitive structures (e.g. brain or eye).

3) When using a drug or implant that can cause serious complications if it is injected accidentally in the wrong place.

4) For injection into an area where the needle can damage other nearby structures.

All four factors are important when it comes to injections of botulinum toxins (as known by the trade name Botox, for example) or similar neurotoxins to the SPG or OG, and some or all of the factors also apply to other medications that one can envisage using in blocking of cranial parasympathetic ganglia. Moreover, since the same or similar requirements arise in many other situations requiring delivery of a substance or insertion of an instrument to a targeted site within the human or animal body then a device and/or method capable of addressing the need for targeting of the cranial parasympathetic ganglia will have numerous other uses and advantages.

As noted above, prior art such as U.S. Pat. No. 7,981,433 discloses administration (topical and by injections) of neurotoxins (e.g. Botox) to parasympathetic (including SPG), trigeminal and occipital nerves in the treatment of headaches, amongst other things.

U.S. Pat. No. 7,981,433 describes an injection technique, specifically a lateral approach, which is a conventional suprazygomatic approach. This approach makes it impossible to accurately deposit substances, since the sphenoid bone will normally obstruct access to the SF and in particular the middle and the posterior compartment and almost always obstruct access to the SPG, making it quite safe, but not applicable for high-precision interventions. Due to the low diffusion rate of botulinum toxin and that the SF mainly contains adipose tissue, a hydrophilic substance will rarely reach its target. There is no consideration in U.S. Pat. No. 7,981,433 of the techniques required to reach other parasympathetic ganglia (most importantly the OG). Thus, there is a significant unmet need for a safe, high-precision system for targeting of cranial parasympathetic ganglia and other similar target sites in the human or animal body.

Image guided techniques are quite complex, especially when performed on awake patient and can move. The surgeon must then compensate continually for the movement of the patient. For procedures performed under sedation and general anaesthesia, the head of the patient can be fixed during a procedure, e.g. by screws to the cranium. For conscious patient this is very painful, and sometimes more importantly an uncomfortable experience, especially combined with a procedure performed towards the head/face that causes pain.

Viewed from a first aspect, the invention provides a guidance device for guidance of surgical interventions on a patient, the surgical interventions requiring an intervention device to surgically enter the body and be directed through body tissues to a target site within the patient's head, the guidance device comprising: a guide piece for guiding the intervention device into the body and directing it to the target site within the patient's head; a mouthpiece arranged to anchor the device in a fixed orientation relative to the patient's upper jaw or lower jaw; and a targeted or targetable mounting supporting the guide piece on the mouthpiece, the mounting being for directing the guide piece in a desired orientation relative to the mouthpiece to thereby direct the intervention device through body tissues to the target site in the patient's head.

The target site may be any location that does not move relative to the patient's upper jaw, or the patient's lower jaw, as the case may be. It should be noted that the first aspect relates to a device for medical surgery and for surgical interventions on the body involving insertion of an intervention device through body tissue to reach a target site. The device of the first aspect is hence not suitable for, or intended for, use in dental surgery and similar procedures involving surgery on the teeth. The device of the first aspect may be for procedures excluding dental procedures, and thus the device may be for procedures excluding operations on the teeth, operations for placing dental implants and so on. In example embodiments the device is for targeted surgical procedures involving surgically entering the body with an intervention device through the tissue of the face or head, which may be for target sites anywhere within the skull, including target sites accessible by entering the patient's body by piercing tissue within the mouth.

By having a mouthpiece that anchors the device at the upper jaw or the lower jaw the invention provides a stable support for the guidance device, such that the desired orientation remains fixed relative to the target site even when the patient moves. Since the guidance device and hence the intervention device can be secured to a structure that does not move relatively in relation to the target site, e.g. the sphenopalatine ganglion with fixation to the upper jaw, then fixation of the head will not be necessary. The upper jaw is fixed relative to most structures of the head/face, with the exception of the lower jaw and structures that move with movements of the lower jaw.

The use of this device to guide an intervention device for high-precision image guided procedures will make the performance of such procedures much easier. Procedures done today by highly specialized and trained surgeons will be available for wide range of medical specialists, including physicians and general practitioners, making the procedures depending on such techniques much more available and in the hand of the diagnostic physician, making treatments easier to implement. Patient care can hence be improved.

In some examples the mouthpiece is arranged to be anchored in a fixed orientation relative to the upper jaw. It is preferred for the mouthpiece to be arranged to be mounted to the patient's upper teeth in order to thereby anchor the device to the upper jaw. The mouthpiece may alternatively or additionally be mounted to the upper gums and/or the roof of the mouth.

In other examples the mouthpiece is arranged to be anchored in a fixed orientation relative to the lower jaw. In this case the mouthpiece may be arranged to be mounted to the patient's lower teeth.

The mouthpiece could optionally be custom made for each patient, or a generic mouthpiece may be used in conjunction with a mouldable inner material for conforming to the patient's upper or lower teeth, gums and/or roof of the mouth. The mouldable inner material may be arranged to set once it has been pressed into the patient's mouth in order to provide a strongly fixed anchoring to the jaw. The mouthpiece may be moulded and fitted to the patient by means of known techniques, such as techniques used in the dental industry. The mouthpiece may be fixed by a mechanical fixing such as a clamp.

One example mouthpiece may be made of heat-sensitive acrylic material, which may be in a gum shield type shape. Another example mouthpiece is ResMed Narval CC.

Advantageously, the device can be a single-use device intended to be disposed of after use. The device can hence be supplied in sterile packaging and used without any special preparation/sterilisation being required, before being disposed of after surgery. Although the device is designed for use with complex and extensive guided surgery systems, the design of the device does not require any expensive materials or moving parts and it can be made disposable without any disadvantage in relation to costs.

The inside of the mouthpiece may be covered with plastic compound, gel or similar to aid good fixation. Adhesive compositions known for use with dentistry may be used.

Optionally, the mouthpiece may be provided with a strap for extending around the patient's head to secure fixation and further hinder movement of the device. This is particularly useful for a mouthpiece secured to the upper jaw, since this does not move relative to the major portion of the patient's head.

The guide piece is attached to the mouthpiece via the mounting and the mounting may be an adjustable coupling that is adjustable so that it can point towards any target site of the face/head. This hence provides a targetable mounting. Such an adjustable coupling may for example include one or more articulated joints allowing for rotation and/or sliding motion of one or more lever arms. In this case the guidance device might typically be used for targeting a site within the patient's head with an approach from outside of the mouth and in front of or to the side of the patient's face. It is preferred for this adjustable coupling to be fully lockable so that it may be fixed in place once the guide piece is in the desired orientation. For example there may be clamps operated by screw devices to prevent movement of each element of the adjustable coupling after the required adjustment has been done. Known joint types used for fixation of surgical devices may be utilised, such as known clamps for joining rods and pins as used in bone fixation. An adjustable coupling may be used, for example, for targeting the SPG or OG via a lateral approach using a device anchored to the patient's upper jaw.

Another possible mounting is a targeted mounting that may be tailor-made for each patient. For example the mounting may be manufactured for a specific target site and a specific approach based on the patient's anatomy. The patient's bone and soft tissue structure may be mapped using imaging techniques, which then allows determination of a required location and orientation for the mounting and for the guide piece held by the mounting. This arrangement might be used for target sites in the patient's head that are approached from the outside of the mouth or from the inside of the mouth. A tailored mouthpiece can be used to target any structure via any required approach, such as the lateral approach to the SPG or OG from a device anchored to the patient's upper jaw as mentioned above. It is, however, considered to have greatest benefit when used for target sites approached from the inside of the mouth.

One example device has a patient tailored mounting arranged for guiding the intervention device toward the SPG via a transpalatine approach, and hence the mounting may be arranged to direct the guide piece toward the inferior opening of the palatine canal. This provides great assistance to the interventionist in accurately targeting the SPG (or other target site) via the transpalatine approach. The penetration depth of the device may be calculated form the medical images, and the length of the device or of the guide piece adjusted accordingly so that an accurate placement of the device is enabled. A trackable needle might be used as the intervention device, as discussed below.

With this type of mounting the guidance device may be arranged to receive the intervention device at the front (by the frontal teeth) and to direct the intervention device to exit within the patient's mouth at a required location and orientation, for example at the inferior opening of the palatine canal and with the right inclination to facilitate the advancement of the intervention device into the canal.

The guide piece may have a varying construction depending on the intervention device that will be used. Preferably the guide piece is arranged to support an intervention device, such as an injection device, and to direct it in a required orientation. In one example the guide piece takes the form of a hollow tube, for example a lumen, which can hence guide a smaller lumen, catheter, needle or similar. The guide piece can be targeted with a required position and angulation relative to the mouthpiece by means of the mounting, for example by means of an adjustable mounting or a patient specific targeted mounting as described above. The guide piece can then direct the intervention device in the desired manner, for example by aligning a needle or other injection device with a required approach to the target site by directing a tube of the guide piece along the required approach.

The correct orientation of the guide piece may be determined based on imaging techniques. This could be done manually by a skilled operator, but it is preferred to have a computer guided device. Hence, in a preferred embodiment the guidance device incorporates a navigational array or an anchor for a navigational array. The navigational array may take the form of a localiser. The navigational array may be arranged for use with a surgical navigation system that may be optical, electromagnetic or any other system. This enables the user to accurately align the guide piece with the desired orientation in order that the guidance device can direct an intervention device towards the target site. The navigational array may be integrated in the mouthpiece. In this case an additional localiser to register the patient will not be necessary. After calibrating the device for an image guided system, it is beneficial to ensure that the device cannot move relative to the patient localizer without the movement being detected. This is particularly important with a targetable mounting that can be moved. Even with a firmly attached mouthpiece and a clamped adjustable coupling there is a chance that a knock or the like will cause some movement.

Advantageously the device may include an indicator for providing a warning of undesirable movement. In one preferred arrangement the guidance device comprises a light source for pointing at the patient. The light source should be attached to the mouth piece and able to be locked in place relative to the guide piece. When the device is calibrated, the light source may be pointed towards a specific anatomical landmark or a mark on the skin made with a standard marker. This means that any inadvertent movement of the guide piece relative to the target site will cause the light to move away from the anatomical landmark or the mark on the skin, making the interventionist aware of the incident. In addition or alternatively, the device may have a camera attached that can register an inadvertent event, e.g. by videometry or simply using a marker registered by the camera, e.g. a fluorescent marker, or illumination from the light source, if present.

The device may incorporate an additional patient localiser mounted on the patient's head, for example via a headband. In this case, with a patient localiser on the patient's head and a navigation array mounted to the mouthpiece then the navigation system may detect any relative movement between the patient localiser and mouthpiece and warn the interventionist. In an alternative arrangement there may be a patient localiser with a light source that can be directed towards the mouthpiece, and the mouthpiece can have a target in place of a navigational array. When the patient is registered the light can be targeted on the mouthpiece and if the light moves away from the target during the procedure this this acts as a warning of patient movement. The target may amplify the light to be easier to detect, for example the target may be a prism and/or may have a fluorescent element. The target may also carry a sensor that will warn the interventionist by setting off an alarm if the light does not hit the target. It will of course be understood that the light source and target or sensor could be reversed, with the light source at the mouthpiece and the target on the patient. Furthermore, the use of light is not the only option and other forms of radiation source and sensor may be used.

The guidance device may comprise a small screen. Images from the navigation system could then be sent to the screen for use in aligning the guide piece with the desired orientation. Movements registered on the navigation system will then be aligned with movement of the device and working instruments, making it easier for a physician without special training performing such procedure. Such screen device may also carry a camera for use as described above.

The guidance device may be used in combination with known intervention devices such as, for example, the device described in WO 2014/037524. It is also possible to make effective use of more basic intervention devices, for example devices not provided with an integrated navigational array or the like, since the guidance device will ensure that the intervention device approaches the patient at the correct angulation for the desired procedure.

In one example, when in use, the distal end of the guide piece may be first placed towards the skin, without entering the body, in the required orientation, and then the intervention device may be advanced through the skin/mucosa toward the target structure. To facilitate this approach, the distal end of the guide piece may be blunt and/or rounded to avoid damage to the patient before the intervention device is advanced. In another example, the guide piece may be used for guidance of the intervention device both outside of the body and inside of the body. Thus, the guide piece may itself be advanced through the skin/mucosa and close to the target structure, to thereby guide the intervention device into close proximity within the body. This approach can be useful if the intervention device is not capable of following the required approach to the target site, for example if it is required to use a device that is not sufficiently rigid, such as a narrow needle. This approach uses similar principles to the device described in WO2014/037524, with the same advantages. The distal end of the guide piece may hence be sharp, and the guide piece arranged to be sufficiently rigid to take the desired approach through the body.

The navigation array may comprise optical markers or electromagnetic location sensors, for example optical reflectors such as reflector balls or electromagnetic coils. Any suitable navigation array system can be used. The navigation array may comprise a plurality of markers located in plane with one another and at known locations relative to the end piece. In one preferred embodiment there are at least three markers, for example there may be four or five markers. The navigation array should be rigidly connected to the guide piece. The guide piece may have a known orientation and size relative to the navigation array, or a calibration sequence may be performed to provide appropriate data concerning the orientation and size of the guide piece relative to the navigation array. A rigid and integrated connection of the navigation array with the guide piece is preferred since this provides the least risk of inaccuracy and in advertent misalignment of the navigation system with the guide piece.

Alternatively, when an anchor point for a navigation array is provided then the anchor point should be arranged for rigid connection of the navigation array to the guide piece. The anchor point may, for example, be for connection to an array of the type supplied under the trade names SureTrack® Universal tracker from Medtronic and Brainlab Instrument Adapter System from Brainlab.

The guidance device may comprise a proximal piece for holding a proximal end of an intervention device used with the guidance device. The proximal piece may be arranged to allow for measurement of and/or control of the depth of insertion of an intervention device into the patient. The proximal piece may be positioned at a proximal end of the guide piece. It is preferred for the proximal piece to comprise parts that are moveable relative to the guide piece and are for fixed connection to the intervention device. Such parts can be used in the manipulation of the intervention device as described below.

In a particularly preferred embodiment the proximal piece comprises one or more clamp(s) for attachment of the intervention device. A clamp or clamps may advantageously be provided on the proximal piece to releasably fix the intervention device in place relative to the guide piece.

When the intervention device has been inserted into the body, guided by the guidance device, to a suitable point with reference to a target site then the intervention device can be operated by manipulation of the proximal end of the intervention device at the proximal piece. For example, the intervention device may be extended from the distal end of the guide piece to move it closer to the target site. When the intervention device includes a needle this allows for highly accurate targeted injection without the risk of damaging the target site. A scale is preferably provided on the proximal piece in order to show the movement of the intervention device, for example how far the intervention device has been inserted into the body.

The proximal piece may comprise two clamps for releasable connection to the intervention device, with one clamp slidable relative to the scale and hence useable to indicate movement of the intervention device. Alternatively, or in addition, the proximal piece may comprise positional markers, e.g. in the case of an optical system, reflectors, for indicating the distance. For example, a positional marker may slide along the proximal piece connected to an associated one of the clamps, which in turn may be for fixed connection to the intervention device during use, so that the positional marker moves along with the intervention device. In a preferred embodiment the proximal piece includes a handle, such as a ring piece, for enabling the user to push or pull the intervention device with the thumb or a finger.

The moveable parts of the proximal piece, which are for connection to the intervention device, may advantageously be connected to the navigation array, or to a further navigational array. This may allow for computer guidance of the depth of insertion of the intervention device.

Advantageously, the guidance device can be used in relation to a target site at any region of the patient's skull that is fixed relative to the upper jaw or the lower jaw, as the case may be. In some preferred embodiments, with the device fixed to the upper jaw, the guidance device is for targeting of the SPG or other of the cranial parasympathetic ganglia, for example the OG. The guidance device may hence be arranged for use with a lumen and/or needle arrangement capable of advancing along the selected approach, which in preferred embodiments is the lateral approach to the ganglion of interest.

The intervention device may be included with the guidance device in order to form a system for guided surgical interventions. The intervention device may include a navigation array, such as a localiser, in order to allow for guided placement of the intervention device, such as guided insertion to a required depth. The intervention device may for example be a needle within a lumen, or it may be a neurostimulator such as a neuromodulator. The intervention device may comprise an implant such as a steroid implant or a drug eluting stent. Steroid implants and drug eluting stents are used for treating sinusitis, and post-operatively to avoid recurrence. Such devices are normally implanted in the ethmoidal sinuses. The implantation technique may in certain cases be quite difficult due to local anatomy. It is described in the literature serious complications due to unintentional insertion in the orbit. Such implants may be implanted with guidance from the device described herein in a safer manner than the prior art techniques.

In some preferred examples the intervention device is a trackable needle, for example an electromagnetic needle, that allows for the operator to track the location of the needle within the body, and in particular a needle that allows the operator to track the location of the needle tip within the body. Such a system can provide additional accuracy in relation to determining when the needle has reached the target site. Possible devices include those marketed under the trade name Aurora by Northern Digital, Inc (NDI) of Ontario, Canada.

The intervention device may also be a needle for core needle biopsy, a needle for fine needle biopsy, an electrode for electric or radiofrequency ablation therapy or a cannula for chemical ablative therapy.

The device may be arranged for use in the method described below in the second aspect and preferred/optional features thereof.

Viewed from a second aspect, the invention provides a method of targeting an intervention device for later surgical intervention on the body, where the device comprises a guide piece for guiding an intervention device; a mouthpiece arranged to anchor the device in a fixed orientation relative to the patient's upper jaw or lower jaw; and a mounting supporting the guide piece on the mouthpiece, the method comprising: non-surgically attaching the mouthpiece of the device to the patient's upper jaw; determining a desired orientation for a guide piece of the device which will enable the guide piece to later guide the intervention device to surgically enter the body and be directed through body tissues to a target site within the patient's head; and setting the orientation of the guide piece relative to the mouthpiece by means of a tailor made mounting or a targetable mounting so that the guide piece is in the desired orientation.

The method may comprise use of the guidance device of the first aspect as described above, and the guidance device used in this method may optionally have features as described in relation to the preferred/optional features of the first aspect. The method hence may be for certain surgical procedures as discussed above.

The invention extends to a method of surgery comprising targeting an intervention device as described above, and then carrying out the surgical intervention including surgically inserting the intervention device into the body and through the body tissue to the target site in the patient's head.

The method may comprise use of a needle as the intervention device and optionally can include guided injection of a pharmacological substance into the body at the target site.

The mouthpiece may be attached to the patient's jaw by securing it to the patient's teeth. The mouthpiece may alternatively or additionally be mounted to the gums and/or the roof of the mouth. Using these parts of the patient's mouth for non-surgical attachment of the mouthpiece means that the mouthpiece can be attached by non-surgical personnel, or even by the patient themselves.

When a targetable mounting is used then preferably the method includes fixing the guide piece in place once it has been adjusted to the desired orientation, for example by clamping and/or locking parts of the mounting to prevent further movement.

In one preferred embodiment the method may comprise navigated insertion of an intervention device toward the SPG along the lateral approach described herein. In the pre-operative planning a standard IGS planning station (e.g. iPlan by Brainlab) may be used to define the best choice of approach (where there is a straight line through soft tissue towards the SPG).

In another preferred embodiment the method may comprise navigated insertion of an intervention device toward the SPG along the transpalatine approach.

In a further example embodiment the method may comprise navigated insertion of an intervention device toward the OG via a transoral approach. The transoral approach may include advancing medial to the mandibular ramus and lateral to the midline of the head, in order to enter the infratemporal fossa and hence target the OG.

The method may be for treating or preventing headache in a patient such as a human in need thereof and may comprise injecting a neuroinhibitory substance such as botulinium toxin in close proximity (i.e. proximally) to the sphenopalatine ganglion or otic ganglion wherein an intervention device in the form of an injection device comprising said neuroinhibitory substance is brought into close proximity to the sphenopalatine ganglion or otic ganglion by inserting said injection device into the patient transnasally, laterally or via a transpalatine approach and the neuroinhibitory substance injected in close proximity to the SPG or OG.

The method may be for treating or preventing rhinitis, rhinosinusitis, Frey syndrome or hypersecretion of tears in a patient such as a human in need thereof and may comprise injecting a neuroinhibitory substance such as botulinium toxin in close proximity to the sphenopalatine ganglion or otic ganglion wherein an injection device comprising said neuroinhibitory substance is brought into close proximity to the sphenopalatine ganglion or otic ganglion by inserting said injection device into the patient transnasally, laterally, transorally or via a transpalatine approach and the neuroinhibitory substance injected in close proximity to the SPG or OG.

The method may be for treating facial pain, including, orofacial pain disorders, myofascial pain disorders, temporomandibular joint disorders, neuropathic orofacial pain, trigeminal neuralgia and oral motor disorders.

The method may include targeting the trigeminal ganglion via a percutaneous approach or transoral approach, for example for treatment of trigeminal neuralgia with glycerol for chemical denervation.

Viewed from a further aspect, the invention provides a computer programme product containing instructions that when executed will configure a computer guided surgery navigation system to determine a required orientation of the guide piece of the guidance device described above when the guidance device has been secured to the patient's upper jaw via the mouthpiece.

Preferably the computer guided surgery system makes use of a navigational array provided on the mouthpiece or on the guide piece.

In a preferred embodiment, the computer programme product configures the image guided surgery navigation system to determine a desired orientation of the guide piece that will target toward the sphenopalatine ganglion (SPG) or the otic ganglion (OG) along a lateral approach. In another preferred embodiment, the computer programme product configures the image guided surgery navigation system to determine a desired orientation of the guide piece that will target toward the sphenopalatine ganglion (SPG) along a transpalatine approach. The system may be configured to determine a desired orientation that will allow the intervention device to be used with a method as described above.

Certain preferred embodiments will now be described by way of example only and with reference to the accompanying drawings in which:

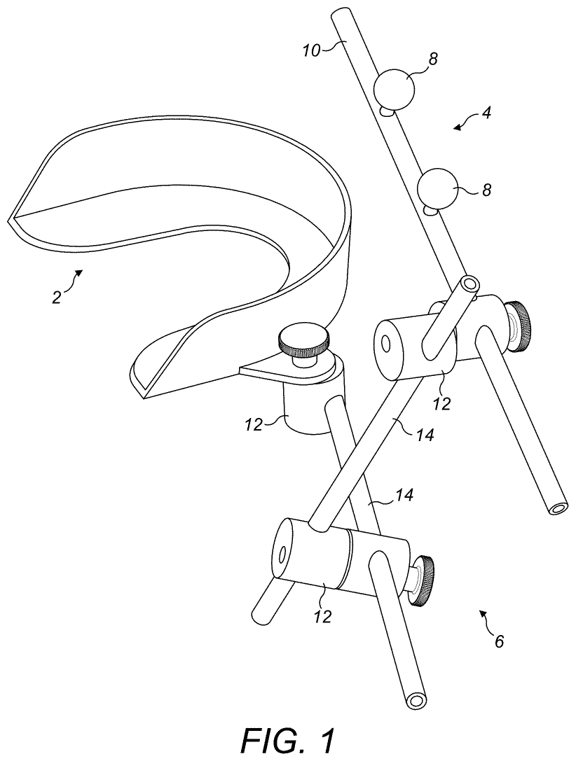

FIG. 1 shows a guidance device in a first perspective view;

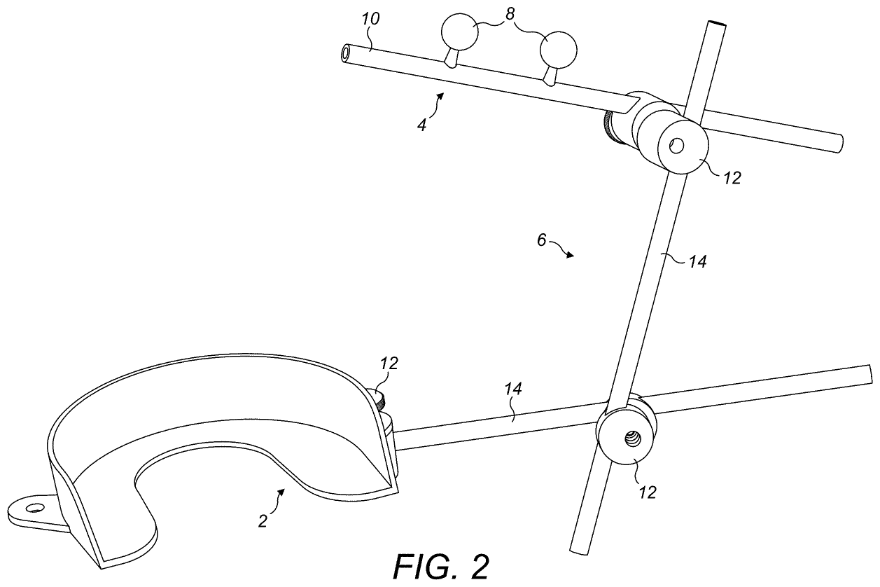

FIG. 2 shows the guidance device of FIG. 1 in a second perspective view;

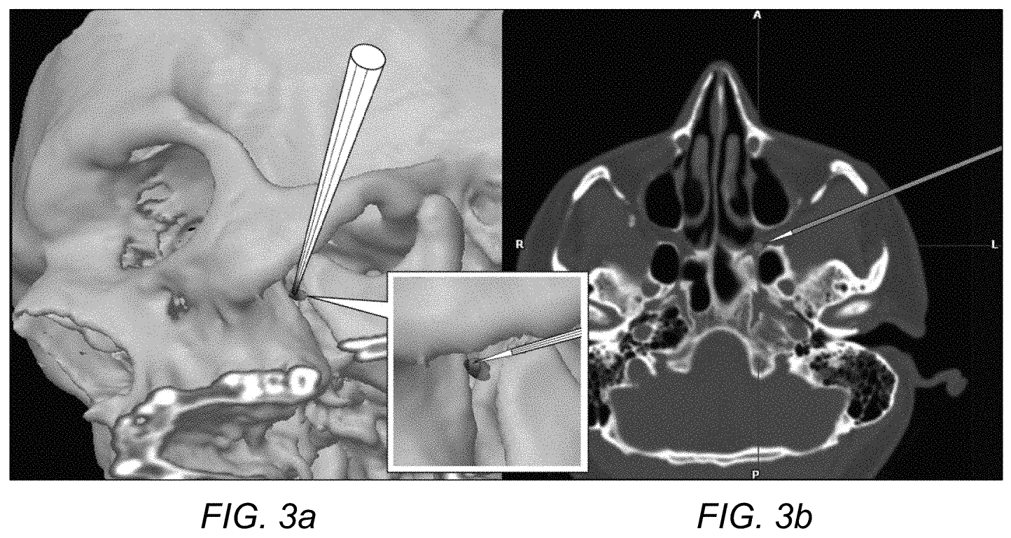

FIGS. 3a and b show the location of the SPG in the head with an intervention device shown approaching the SPG laterally;

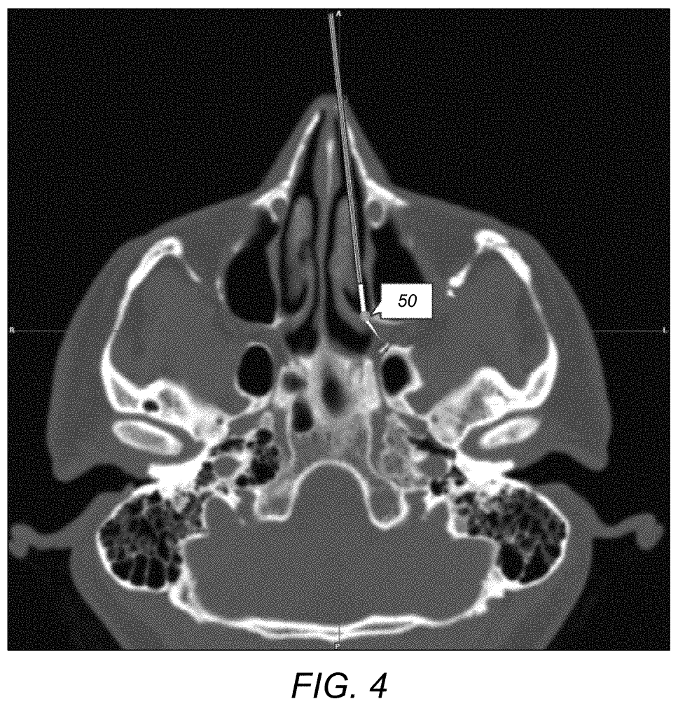

FIG. 4 shows the transnasal approach with an intervention device having an angled tip;

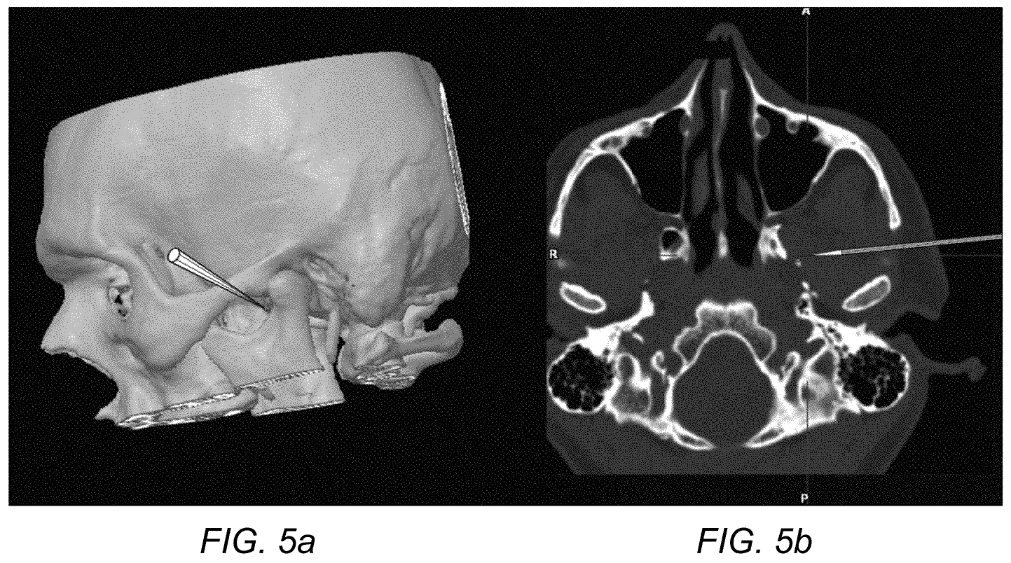

FIGS. 5a and b show a lateral approach to the OG;

FIG. 6 shows a transnasal approach to the OG, this approach being defined by a straight line;

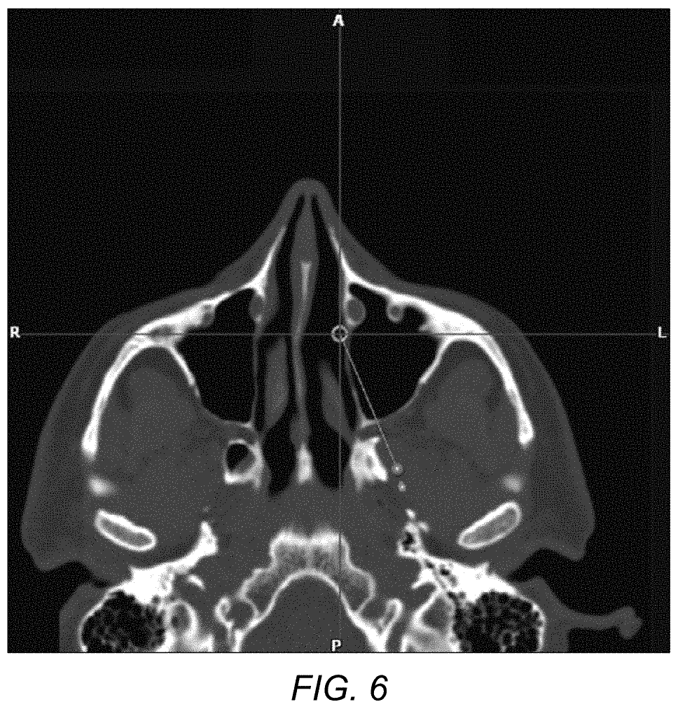

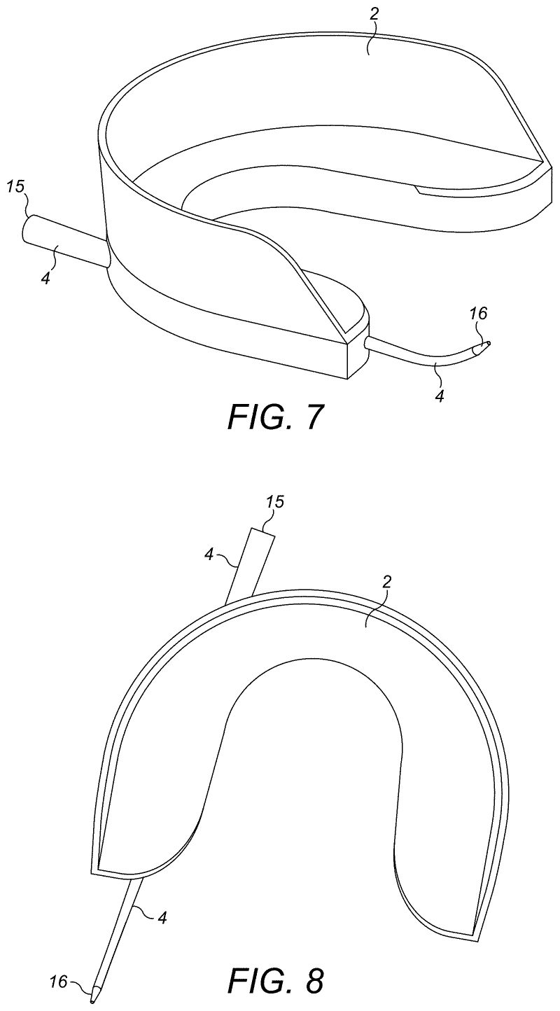

FIG. 7 shows an alternative guidance device in perspective view; and

FIG. 8 shows the device of FIG. 7 viewed from above.

As shown in FIGS. 1 and 2 a guidance device is provided with a mouthpiece 2, a guide piece 4 and an adjustable coupling 6. The mouthpiece 2 in this embodiment has a shape similar to a gum shield, and can be attached to the patient's upper teeth via dental adhesive products, for example. The guide piece 4 has optical markers 8 forming a navigational array for use with an image guided navigation system. It will be understood that other forms of markers could be used. The guide piece 4 includes a lumen/guide tube 10 to which the navigational array 8 is attached. The lumen 10 can be aligned with a desired orientation so that an intervention device, such as a needle for example, can be inserted along the lumen toward a target site in the patient's head. The target site can be any location that is fixed relative to the upper jaw, for example it may be the SPG targeted via a lateral approach. The adjustable coupling 6 has clamps 12 that releasably attach the lumen 10 to the mouthpiece 2 via two rods 14. It will be understood that there may be differing numbers of clamps 12 and rods 14. Alternative designs of adjustable coupling 6 are also possible. Provided an adjustable and preferably lockable movement of the lumen 10 can be permitted then any type of joint can be used in the adjustable coupling 6.

With this embodiment the mouthpiece is first fixed to the patient via the patient's teeth. Then the clamps 12 are loosened to permit a sliding and rotating motion of the rods 12 in order to allow adjustment of the lumen 10 into a desired orientation. Computer guided surgery systems can be used to guide the adjustment process. When the lumen 10 is at the desired angulation and location then the clamps 12 can be tightened to lock the mechanism. The interventionist may then carry out the required intervention with precision, since even if the patient moves the guide piece 4 will remain in the required orientation relative to the target site.

As described above, there may be a light source of a camera (not shown) attached to the device, preferably at the mouthpiece 2, in order to provide a visible alert of undesired movement of the guide piece 4 and/or mouthpiece 2 away from the desired orientation. The device can also include a screen (not shown) on a tablet or smart phone, with the screen being arranged to show guidance information from a computer navigation system.

FIGS. 7 and 8 show another guidance device. This has a similar mouthpiece 2, but the guide piece 4 has a different design. This example uses a tailored guide piece 4, where the orientation of the guide piece 4 is set based on imaging data showing the patient's anatomy. In this example the guide piece 4 is arranged to target the SPG via a transpalatine approach. The mouthpiece 2 is for fixation to the patient's upper teeth to thereby fix the device (and the guide piece 4) relative to the upper jaw. The device is then fixed relative to the SPG and also to the transpalatine canal. The guide piece 4 has a proximal end 15 that will protrude out of the patient's mouth and can receive an intervention device, for example a needle. The guide piece 4 also has a distal end 16 that will be within the patient's mouth and has an orientation specifically tailored to the patient in order to direct the intervention device along the palatine canal toward the SPG.

It will be understood that either of the example devices could be readily adapted for mounting to the lower teeth in order to hence allow for targeting of sites that are fixed relative to the lower jaw.

The proposed device leads to numerous advantages:
It is not necessary to fixate the patient head and this enables procedures formerly performed under sedation/general anaesthesia to be performed on a conscious patient, leading to procedures with less complication and lower cost.
The guide is attached to the patient without fixating the head, and will despite movement of the head point directly towards the target structure. The interventionist therefore can use both hands to handle the working device, syringe, pharmacological substances etc.
The procedures will be much easier to perform and therefore within the reach of physicians and GPs, making the procedure more available to patients.
The light source and/or camera will warn the interventionist if the device inadvertently moves relative to the target, making procedures more accurate and safer for the patient.
The screen on device will make the image guided procedure more user-friendly and lower the threshold for performing the procedure for professionals not trained for conventional image guided procedure.

The devices described above makes it safer to use a lateral or transpalatine approach targeting the SPG, as well as other procedures (for example, targeting the OG), significantly lowering the risk of complications such as tissue destruction of adjacent structures by the very instrument at use or by adverse events due to misjudged placement of the needle while injecting the pharmacological substance. At the same time the positioning of the injection will be highly accurate, making it feasible to use small volumes with minimal possibilities of diffusion into adjacent structures. Such a precision also ensures optimal delivery of the pharmacological substances and therefore optimal treatment effect.

In the case of electromagnetic navigation, which can be used as an alternative or in addition to optical navigation, a coil can be embedded in the guide piece 4.

A possible advantageous use of the device is the injection of neuroinhibitory substances such as botulinum toxin in close proximity to the SPG or OG. Note that the injection device should not penetrate the SPG or OG. The injection is achieved in order to treat or prevent headache and may be achieved without damage to surrounding critical structures within the head. A neuroinhibitor is defined as any substance that affects transmission in a neural structure, resulting in any change of transmission, which may decrease or increase the neural activity. The neuroinhibitory substance is preferably a neurotoxin.

By delivery of the active substance in close proximity (proximally) to the sphenopalatine ganglion or otic ganglion means that the botulinum toxin or other neuroinhibitory substance in question is delivered so that it causes the desired technical effect, e.g. the prevention of treatment of headache etc. Ideally therefore the neuroinhibitory substance is injected to within 5 mm of the SPG or OG, preferably within 4 mm, such as within 3 mm, especially within 2 mm. Ideally injection of the active ingredient takes place 2 mm or less form the target SPG or OG.

The injection of the neuroinhibitor occurs laterally, transnasally, transorally or via the transpalatine approach in order to ensure that a safe, close injection of the neuroinhibitor is achieved. The terms laterally, infrazygomatically, transnasally and transorally are terms of this art.

The term infrazygomatic therefore requires that the injection takes place inferior to the zygomatic arch on either side of the mandibula, typically anterior or through the mandibular notch.

The term transnasally defines an injection route which involves advancing the needle through the nasal cavity. Targeting the SPG this route will further violate the lateroposterior boundary of the nasal cavity, constituting the medial boundary of the SF.

The preferred transoral approach is for the OG and hence includes advancing medial to the mandibular ramus and lateral to the midline of the head, in order to enter the infratemporal fossa and hence target the OG.

Targeting the OG transnasally involves advancing through the maxillary ostium and the maxillary sinus, violating the back wall of the maxillary sinus, advancing on the lateral aspect of the lateral pterygoid plate. The OG is located in the infratemporal fossa, the SPG in the sphenopalatine fossa.

It is preferably the case that access to the SPG or OG from the outside of the body is achieved laterally or transnasally by insertion of the injection device such that the device defines a straight line between SPG or OG (or more specifically the point proximal to the SPG and OG where active substance release will occur) and the point at which the external skin or mucosa is penetrated. This is illustrated in FIGS. 13, 15 and 16. FIG. 14 shows an alternative preferred approach where the end piece of the device has a curved tip enabling the needle to be directed toward the SPG or OG at an angle from the main axis of the lumen. The device punctures the wall of the nasal cavity at puncture site 50 and the angled tip directs the needle toward the target site.

The lateral approach therefore allows the injection device to pass through the skin and then soft tissue to the SPG or OG. That can be achieved in a straight line and hence with a straight injection device. That means that the injection can be targeted very accurately in close proximity to the SPG or OG. This method of administration allows application under local anaesthetic.

Where the injection takes place transnasally the route involves passing through the nasal mucosa and the sphenopalatine foramen or the perpendicular plate of the palatine bone to reach the SPG. Injection is not therefore lateral (via the cheek) but preferably involves a straight line from the injection point to the SPG. Transnasal route to reach the OG involves advancing through the maxillary ostium and the maxillary sinus, violating the back wall of the maxillary sinus, advancing on the lateral aspect of the lateral pterygoid plate. This involves a straight line from the injection site to the OG. These methods may require general anaesthesia.

Using the transpalatine approach, as the guide piece 4 is accurately targeted toward the inferior opening of the palatine canal then high-precision interventions targeting the SPG are possible, unlike in the prior art. The oral cavity communicates with the sphenopalatine fossa through the greater traspalatinal canal. The inferior opening is situated on the medial side of the second molar and the length of the canal is on average 25 mm. The canal transmits the descending palatine artery and vein, and the greater and lesser palatine nerves.

The injection described above can be used in the treatment or prevention of headaches, in particular any kind of primary headache or secondary headache. The treatment or prevention may relate therefore to cluster headaches, migraine, tension-type headache, short lasting unilateral neuralgiform headache with conjunctival injection and tearing/cranial autonomic features (SUNCT/SUNA), hemicrania continua or paroxysmal hemicrania.

Paroxysmal hemicrania is a primary headache disorder involving frequent attacks of unilateral, peri-orbital and temporal pain typically lasting less than 30 minutes. The pain can be associated with conjunctival injection, lacrimation, nasal congestion, rhinorrhea, ptosis and eyelid edema.

SUNCT/SUNA is a primary headache disorder characterized by multiple attacks of unilateral, peri-orbital and temporal pain typically lasting less than 2 minutes. The pain is associated with conjunctival injection, lacrimation, nasal congestion, rhinorrhea, and eyelid edema. This headache may be associated with trigeminal neuralgia.

Hemicrania continua is a primary headache disorder characterized by a strictly unilateral headache responsive to Indomethacin. The pain is associated with conjunctival injection, lacrimation, nasal congestion, rhinorrhea, ptosis, and eyelid edema.

It will be appreciated that the term treatment here refers to reduction in pain experienced by a patient and/or a reduction in the frequency in which headache occurs. The term prevention means preventing headaches occurring, e.g. as frequently as before.

The neuroinhibitory substance is one which is capable of preventing or treating headache when administered in close proximity to the SPG or OG. Suitable inhibitors include Botulinum toxin, Tetanus neurotoxin, (which is produced by Clostridium tetani), Staphylococcal alpha-toxin, and acylpolyamine toxins (e.g. AR636 and AG489).

In general the therapeutic modality used to treat and/or prevent headache is a presynaptic neurotoxin. “Presynaptic neurotoxin” as used herein refers to those neurotoxins and their derivatives which are known to produce localized, reversible flaccid paralysis of musculature in mammals which does not result in degeneration of muscle or nervous tissue.

It is preferred however if the inhibitor is botulinum toxin. This is a protein and neurotoxin produced by the bacterium Clostridium botulinum and is commercially available. It is preferred if the botulinum toxin is of types A, B, C, D, E, F or G, such as Botulinum toxin type A. Botulinum toxin may for example be administered in the manner and form described in U.S. Pat. No. 7,981,433

The frequency of the injections needed may be every 3 to 8 months but will be patient dependent.

Whilst the method described above is in relation to the administration of neuroinhibitory substances such as botulinium toxin, the method of injection and device discussed here can be used for the injection of other active substances such as local anaesthetics (e.g. lidocaine or marcain) and corticosteroids (e.g. triamcinolone). The method and device may be used to inject a local anaesthetic or corticosteroid for use in a method for treating or preventing headache, rhinitis, rhinosinusitis, Frey syndrome or hypersecretion of tears/lacrimation comprising injecting said substance in close proximity to the sphenopalatine ganglion or otic ganglion wherein an injection device comprising said substance is brought into close proximity to the sphenopalatine ganglion or otic ganglion by inserting said injection device into the patient transnasally or laterally and the substance injected in close proximity to the SPG or OG.

Various example procedures that can advantageously make use of the guidance device described above are set out below and FIGS. 3a through 6 illustrate the locations of the SPG and OG along with possible approaches for interventions on the SPG or OG as discussed above.

### EXAMPLE 1

A female patient with refractory hemicrania continua was treated via injection of Botox around the SPG. Due to an occipital neurostimulator MRI was contraindicated and identification of SPG on MRI was not possible. Preoperatively the calculated position of the SPG was marked on a CT scan with 1 mm slides. On the navigation planning system a preplanned puncture site and trajectory was made. On the symptomatic side a navigable needle guide was advanced through the sphenopalatine foramen and towards the SPG. The needle was passed through the guide and the tip of the needle was confirmed to be 1 mm from the SPG by the navigation system while 75 IU botulinum toxin type A was injected.

Over a period of two months prior to the treatment the patient had an average headache intensity of 8.1 (scale 1-10) and normally experienced from one to four headache attacks daily. From 4 to 10 weeks after the treatment the patient had not a single attack during the whole period and the average headache intensity was 6.3. The patient also did not experience any complication during 4 months follow-up.

### EXAMPLE 2

The patient was a male that presented with a prevertebral mass close to the atlas (Cl) seen on MRI. He had formerly been treated for pulmonary cancer histologically classified as adenocarcinoma. After a clinical assessment it was concluded that the tumor was not available for conventional procedures for a histological diagnosis. Using a navigable guide with an optical navigation system and a transoral approach it was possible to do a fine needle biopsy of the tumor deep in the neck to confirm the suspicion of a pulmonary metastasis.

### EXAMPLE 3

A female patient with refractory chronic cluster headache was treated via injection of lidocaine around the OG. Preoperatively the calculated position of the OG was marked on a CT scan with 1 mm slides. On the navigation planning system a pre-planned puncture site and trajectory was made. On the symptomatic side a navigable needle guide was advanced through the maxillary ostium and the back wall of the maxillary sinus, and then at the lateral aspects of the lateral pterygoid plate to the OG. 5 ml of lidocaine 20 mg/ml was injected. The patient had a short relief of the headache as expected using short-acting local anaesthetic.

### EXAMPLE APPLICATIONS

The advantages for interventions targeting the SPG will also arise when using the device for IGS in other parts of the patient that are fixed relative to the upper jaw for indications such as injections, biopsies, punctures, aspiration, ablation therapy, and for positioning of electrodes, catheters, radioactive seeds and implants. The design of the intervention device use with the guidance device can be varied as required. For example, it may be advantageous to use a similar device with an alternative tip design or a different length of end piece, depending on the characteristics of the target site, the approach available and the procedure that is to be carried out. The guidance device may thus be utilised for procedures to address numerous medical conditions. Procedures that the guidance device can be used for include:
Injections
   Any pharmacological substance
   Neuroexcitatory agent
   Neuroinhibitory agents
   Botulinum toxin, any type
   Staphylococcal alpha-toxin
   Tetanus neurotoxin
   Acylpolyamine toxins
Core needle biopsy and fine needle biopsy
   Head and neck area
      Intracranially
      Extracranially
         Retropharyngeal space
         Parapharyngeal space
         Skull base
         Deep regions of the face
         Any region of the face
      In the vicinity of the columna
      In the vicinity of bone
Puncture and aspiration
   Evacuation of cystic structures and fluidic compartment for diagnosis and therapy of the head and neck region
Ablation therapy
   Any nerve or neural structure, intracranially and extracranially
   Ablation of normal tissue to reduce volume and/or increase stiffness
   Ablation of tumour tissue
Positioning of electrodes, catheters, implants, electrophysiological measurements, radioactive seeds
Endoscopy and/or pointer procedures
   Flexible or rigid endoscope may be attached to the device
   Any procedure in an open cavity that requires endoscope or pointer
      Paranasal sinusis
      Nasal cavity
      Farynx
      Larynx
Facet blocks

The device can be used in the treatment of conditions including:
Headache
   Migraine
   Cluster headache
   Tension-type headache
   Trigeminal Autonomic Headache
   SUNCT
   Hemicrania Continua
   Paroxysmal hemicrania
   Any kind of primary headache
   Any kind of secondary headache
Rhinitis
   Allergic rhinitis
   Vasomotor rhinitis
   Rhinitis medicamentosa
   Polypous rhinitis
   Any kind of non-structural rhinitis
Rhinosinusitis
   Without polyps
   With polyps
   Any kind of rhinosinusitis
Hypersecretion of tears/excessive lacrimation
   Any disease with hypersecretion of tears
Frey syndrome/auriculotemporal syndrome/gustatory sweating
Tinnitus
   Objective tinnitus
   Subjective tinnitus
Neck and back pain/syndromes
   Spinal nerve/root blocks
   Spinal taps
Post traumatic neck pain
Cervical disc disorders
Myofacial neck pain
Rheumatic and arthritic disorders
Neuromuscular disorders
Facial pain
   orofacial pain disorders
   myofascial pain disorders
   temporomandibular joint disorders
   neuropathic orofacial pain
   trigeminal neuralgia
   oral motor disorders

Whilst the indications and examples above primarily relate to conditions of the human body the device can of course also be utilised for interventions on the animal body.

1. A guidance device for guidance of surgical interventions on one patient based on patient specific imaging data for the anatomy of the one patient, the device comprising:
a mouthpiece configured to be anchored to said one patient's upper jaw; and
a hollow tube, fixed with respect to the mouthpiece, for guiding an intervention device and directing it to a target site within said patient's head, the hollow tube having a proximal end that can receive the intervention device and that is oriented to protrude out of said one patient's mouth when the mouthpiece is anchored to said one patient's upper jaw, and a distal end that will be positioned within said one patient's mouth when the mouthpiece is anchored to said one patient's upper jaw, the distal end having a customized orientation with respect to the mouthpiece based on the imaging data showing said one patient's anatomy such that when the mouthpiece is anchored to said one patient's upper jaw the distal end will direct the intervention device out of the distal end and along said one patient's palatine canal toward the target site within said one patient's head via a transpalatine approach as the intervention device is guided through the hollow tube.

2. The guidance device as claimed in claim 1, wherein the mouthpiece is configured to be anchored to said one patient's upper teeth.

3. The guidance device as claimed in claim 1, wherein the mouthpiece is configured to be anchored to said one patient's upper gums.

4. The guidance device as claimed in claim 1, wherein the mouthpiece is configured to be anchored to the roof of said one patient's mouth.

5. The guidance device as claimed in claim 1, wherein the mouthpiece is configured to be anchored to said one patient's upper jaw with at least one clamp.

6. The guidance device as claimed in claim 1, comprising a mouldable inner material.

7. The guidance device as claimed in claim 1, wherein the target site within said one patient's head is the patient's SPG.

8. The guidance device as claimed in claim 1, wherein the device is a single-use device that is disposed of after use.

9. A system for guided surgical interventions comprising a guidance device as claimed in claim 1 and an intervention device.

10. The system as claimed in claim 9, wherein the intervention device is a needle.

11. The system as claimed in claim 9, wherein the intervention device is a trackable needle that allows for an operator to track the location of the needle within said one patient's body.

12. The system as claimed in claim 9, wherein the intervention device includes a navigation array to allow for guided placement of the intervention device to a required depth within said one patient.

13. A method of targeting an intervention device for surgical intervention on the body using a guidance device, the guidance device comprising a mouthpiece configured to be anchored to a patient's upper jaw, and a hollow tube fixed with respect to the mouthpiece and for guiding the intervention device and directing it to a target site within the patient's head, the hollow tube having a proximal end that can receive the intervention device and that is oriented to protrude out of the patient's mouth when the mouthpiece is anchored to the patient's upper jaw, and a distal end that will be positioned within the patient's mouth when the mouthpiece is anchored to the patient's upper jaw, the distal end having a customized orientation with respect to the mouthpiece based on imaging data showing a particular patient's anatomy such that when the mouthpiece is anchored to the particular patient's upper jaw the distal end will direct the intervention device out of the distal end and along the particular patient's palatine canal toward the target site within the particular patient's head via a transpalatine approach when the device is guided through the hollow tube; the method comprising:
non-surgically anchoring the mouthpiece to the particular patient's upper jaw;
inserting the intervention device into the proximal end of the hollow tube; and
guiding the intervention device through the hollow tube, out of the distal end and along the particular patient's palatine canal to the target site within the particular patient's head via the transpalantine approach.

14. The method as claimed in claim 13, wherein the mouthpiece is non-surgically anchored to the particular patient's upper jaw by anchoring it to the particular patient's upper teeth.

15. The method as claimed in claim 13, wherein the intervention device is a needle.

16. The method as claimed in claim 13, wherein the intervention device is a trackable needle that allows for an operator to track the location of the needle within the particular patient's body.

17. The method as claimed in claim 13, wherein the intervention device includes a navigation array to allow for guided placement of the intervention device to a required depth within the particular patient.

18. The method as claimed in 13, wherein the target site in the particular patient's head is that patient's SPG.

19. The method as claimed in claim 13, further comprising injecting a pharmacological substance at the target site within the particular patient's head with the intervention device.

20. A method of forming a guidance device for surgical intervention on the body, the method comprising:
obtaining imaging data of a particular patient's anatomy for determining a route along the particular patient's palatine canal toward a target site within that patient's head via a transpalatine approach;
forming a mouthpiece configured to be anchored to the particular patient's upper jaw;
forming a hollow tube configured for guiding an intervention device and directing it to the target site within the particular patient's head, the hollow tube having a proximal end that can receive an intervention device and a distal end that has a customized orientation relative to the proximal end based on the imaging data of the particular patient's anatomy; and
attaching the hollow tube to the mouthpiece in a customized orientation based on the imaging data of the particular patient's anatomy such that when the mouthpiece is anchored to that particular patient's jaw the proximal end of the hollow tube protrudes out of the particular patient's mouth and the distal end of the hollow tube is positioned within the particular patient's mouth and is configured to direct the intervention device out of the distal end and along the particular patient's palatine canal toward the target site within the particular patient's head via a transpalatine approach when the intervention device is guided through the hollow tube.

21. The method of claim 20, wherein the target site within the particular patient's head is that patient's SPG.
